(19) United States
(12) Patent Application Publication
(10) Pub. No.: US 2017/0107473 A1
(43) Pub. Date: Apr. 20, 2017

(54) CONTROL OF CARBON DIOXIDE LEVELS AND PH IN SMALL VOLUME REACTORS

(71) Applicants: Massachusetts Institute of Technology, Cambridge, MA (US); Sanofi, Paris (FR)

(72) Inventors: Shireen Goh, Singapore (SG); Rajeev Jagga Ram, Arlington, MA (US); Michelangelo Canzoneri, Dusseldorf (DE); Horst Blum, Kriftel (DE)

(73) Assignees: Massachusetts Institute of Technology, Cambridge, MA (US); Sanofi, Paris (FR)

(21) Appl. No.: 15/161,112

(22) Filed: May 20, 2016

Related U.S. Application Data
(63) Continuation of application No. 14/063,967, filed on Oct. 25, 2013.
(60) Provisional application No. 61/869,111, filed on Aug. 23, 2013.
(60) Provisional application No. 61/719,027, filed on Oct. 26, 2012.

(30) Foreign Application Priority Data
Oct. 23, 2013 (EP) EP13306462.6

(51) Int. Cl. C12M 1/34; C12M 1/00; B01L 3/00; C12M 1/04

(52) U.S. Cl. CPC C12M 41/34; C12M 23/24; C12M 23/26; C12M 23/34; C12M 29/04; C12M 41/26; C12M 41/32; B01L 3/502723; C12M 41/44

(57) ABSTRACT

Strategies to control the level of dissolved carbon dioxide (CO₂) concentrations and/or pH in small volume reactor chambers, and associated articles, systems, and methods, are generally provided. In certain embodiments, the reactor chambers can be configured to contain at least one biological cell.

### RELATED APPLICATIONS

This application claims priority to U.S. Provisional Patent Application Ser. No. 61/719,027, filed Oct. 26, 2012, and entitled “Control of Carbon Dioxide Levels and pH in Small Volume Reactors”; U.S. Provisional Patent Application Ser. No. 61/869,111 filed Aug. 23, 2013, and entitled “Control of Carbon Dioxide Levels and pH in Small Volume Reactors”; and European Application No. 13306462.6, filed Oct. 23, 2013, and entitled “Control of Carbon Dioxide Levels and pH in Small Volume Reactors,” each of which is incorporated herein by reference in its entirety for all purposes.

### TECHNICAL FIELD

Systems and methods for the control of carbon dioxide levels and pH within small volume reactors are generally described.

### BACKGROUND

There is currently a great deal of interest in developing small volume bioreactors for growing cells, for example, for biopharmaceutical production. Controlling carbon dioxide levels and pH in such reactors can be challenging. Even small amounts of acid, base, and/or carbon dioxide to a small-scale bioreactor can lead to large relative shifts in carbon dioxide levels and/or pH, which can adversely impact bioreactor operation. Improved systems and methods for controlling carbon dioxide levels and pH in such reactors are therefore desirable.

### SUMMARY

Control of carbon dioxide levels and pH within small volume reactors, as well as related systems and methods, are generally described. In certain embodiments, control of carbon dioxide levels and pH within liquid growth medium within a bioreactor, such as a reactor configured to grow one or more types of biological cells, is described. The subject matter of the present invention involves, in some cases, interrelated products, alternative solutions to a particular problem, and/or a plurality of different uses of one or more systems and/or articles.

In one aspect, a bioreactor system is provided. In certain embodiments, the bioreactor system comprises a reactor chamber having a volume of equal to or less than about 50 milliliters and containing a liquid growth medium including at least one biological cell and a buffer and a gaseous headspace containing carbon dioxide above the liquid growth medium, a first inlet connecting a source of carbon dioxide gas to the gaseous headspace, and a second inlet connecting a source of an alkaline liquid to the liquid growth medium.

In some embodiments, the bioreactor system comprises a reactor chamber having a volume of equal to or less than about 50 milliliters and containing a liquid growth medium including at least one biological cell and a buffer and a gaseous headspace containing carbon dioxide above the liquid growth medium, a first inlet connecting a source of carbon dioxide gas to the gaseous headspace, and a sensor within the reactor chamber configured to determine the concentration of carbon dioxide and/or pH within the liquid growth medium.

According to certain embodiments, a method of operating a bioreactor is described. In some embodiments, the method comprises providing a reactor chamber having a volume of equal to or less than about 50 milliliters and containing a liquid growth medium including at least one biological cell and a gaseous headspace containing carbon dioxide above the liquid growth medium, and operating the reactor such that the k_{L}a of carbon dioxide between the headspace and the bulk of the liquid medium is at least about 0.1 hours⁻¹ and less than about 15 hours⁻¹. In some such embodiments, the k_{L}a of carbon dioxide between the headspace and the bulk of the liquid medium is at least about 0.1 hours⁻¹ and less than about 10 hours⁻¹.

In certain embodiments, the method comprises providing a reactor chamber having a volume of equal to or less than about 50 milliliters. The reactor chamber contains, in some embodiments, a liquid growth medium including at least one biological cell, and a gaseous headspace containing carbon dioxide above the liquid growth medium. In some embodiments, the method comprises transporting a gas containing carbon dioxide to the gaseous headspace, and transporting an alkaline liquid to the liquid growth medium. In some embodiments, the osmolarity of the liquid growth medium is substantially constant during the step of transporting the gas.

In certain of the embodiments mentioned above, the biological cell can be a eukaryotic cell.

According to certain of the embodiments mentioned above, a partial pressure of the carbon dioxide above the liquid growth medium can be between about 0% and about 20%.

According to some of the embodiments mentioned above, a total pressure of the gaseous headspace is from about 0 psi to about 15 psi.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a cross-sectional schematic illustration of a reactor system, according to one set of embodiments;

FIGS. 2A-2C are, according to certain embodiments, cross-sectional schematic illustrations of a reactor chamber and a mode of operating the same;

FIG. 3 is a bottom-view cross sectional schematic illustration of a reactor system including a plurality of reactor chambers arranged in series, according to some embodiments;

FIG. 4 is a cross-sectional schematic illustration of a reactor system, according to certain embodiments;

FIG. 5 is a cross-sectional schematic illustration of a gas manifold for a reactor system, according to one set of embodiments;

FIG. 6 is a cross-sectional schematic illustration of a gas manifold for a reactor system, according to some embodiments;

FIG. 7 is a photograph of a reactor system, according to certain embodiments;

FIG. 8 is a plot of phase difference versus frequency, according to one set of embodiments;

FIG. 9 is a plot of phase difference versus modulation frequency, according to some embodiments.

FIG. 10 is a calibration plot for carbon dioxide, according to certain embodiments;

FIG. 11 is a gas transfer plot obtained using an oxygen sensor, according to one set of embodiments;

FIG. 12 is a gas transfer plot obtained using a carbon dioxide sensor, according to one set of embodiments; and

FIG. 13 is a plot of pH versus percent of carbon dioxide in the gas mix of an exemplary reactor system, according to one set of embodiments.

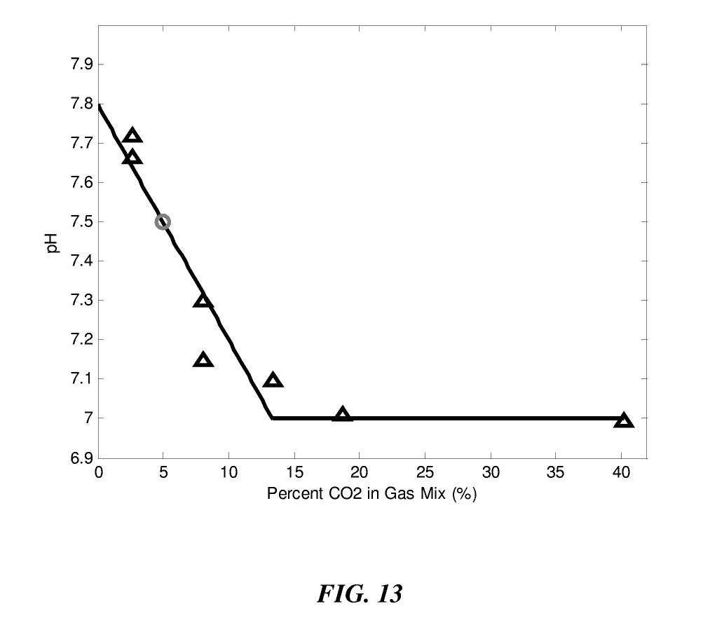

### DETAILED DESCRIPTION

Strategies to control the level of dissolved carbon dioxide (CO₂) concentrations and/or pH in small volume reactor chambers, and associated articles, systems, and methods, are generally provided. In certain embodiments, the reactor chambers can be configured to contain at least one biological cell. For example, the reactor chambers can be bioreactor, such as microbioreactors. The cells within the reactor chamber can be suspended in a liquid medium, such as any common cell growth medium known to those of ordinary skill in the art. The cell growth medium may contain, for example, essential amino acids and/or cofactors. In some embodiments, the reactor chamber comprises a gaseous headspace above the liquid growth medium.

Certain embodiments relate to the control of pH and CO₂ levels in relatively small reactors, including reactors with volumes of less than about 50 milliliters. In certain embodiments, the reactor chamber has an aspect ratio of less than about 10 (or less than about 8, such as between about 5 and about 8), as measured by dividing the largest cross sectional dimension of the chamber by the smallest cross-sectional dimension of the chamber. It has unexpectedly been discovered that pH and dissolved CO₂ levels can be controlled in such small reactors while achieving performance (including oxygen and CO₂ mass transfer rates) similar to those observed in larger scale reactors.

In certain embodiments, the liquid growth medium contains a buffer, such as a bicarbonate buffer solution, to keep the CO₂ and pH levels relatively constant within the liquid growth medium. In certain embodiments, the partial pressure of CO₂ in the gaseous headspace can be increased, which can result in a decrease in the pH of the liquid medium and an increase the dissolved CO₂ level in the liquid medium. In some embodiments, the partial pressure of the CO₂ in the gaseous headspace can be decreased, which can result in an increase in the pH and a reduction in the dissolved CO₂ level in the liquid medium.

In some embodiments, an alkaline material can be transported into the liquid medium to control pH of the liquid. For example, in certain embodiments, a base (e.g., an alkaline liquid) such as a bicarbonate-based base (e.g., a bicarbonate solution) can be added to the liquid medium, which can increase the pH of the liquid medium and decrease the dissolved CO₂ concentration within the liquid medium. In some embodiments (e.g., in certain embodiments in which one wishes to change the pH of the liquid medium while keeping the dissolved CO₂ relatively constant), an acidic material (e.g., an acidic liquid) can be transported to the liquid medium, optionally in conjunction with either or both of addition of an alkaline material (e.g., a liquid base) and/or change in the partial pressure of CO₂ in the gas headspace.

The reactor chamber can include one or more sensors. The sensors can be used, for example, to aid in the control of pH and/or CO₂ levels within the liquid medium. In certain embodiments, the reactor chamber contains at least a CO₂ and/or a pH sensor in contact with the liquid within the chamber.

In some embodiments, the liquid within the reactor chamber can be mixed and/or aerated. In certain embodiments, the reactor chamber can include a liquid sub-chamber (in which the liquid growth medium can be contained) and a gas sub-chamber. The liquid and the gas sub-chambers can be separated, in certain embodiments, by a moveable wall (e.g., a flexible membrane). The moveable wall can be permeable to at least one gas (e.g., oxygen and/or carbon dioxide), in some embodiments. As described in more detail below, in some embodiments, mixing and aeration within the reactor chamber can be achieved by arranging multiple reactor chambers in series and pressurizing one or more of the gas sub-chambers, which can result in the deflection of the moveable wall adjacent to the pressurized sub-chamber and at least partial evacuation of the liquid in the underlying sub-chamber to other reactor chambers within the series. Mixing and aeration within such reactors can also be achieved via the diffusion of gas from the gaseous headspace into the liquid either through direct contact (e.g., in cases in which the gas and liquid components are not separated by a moveable wall) or through a membrane that is permeable to CO₂ and/or other gasses (e.g., in cases in which the gas and liquid components are separated by a moveable wall). Reactors employing such mixing and aeration methods are described, for example, in U.S. patent application Ser. No. 13/249,959 by Ram et al, filed Sep. 30, 2011, and entitled “Device and Method for Continuous Cell Culture and Other Reactions” and U.S. Patent Application Publication No. 2005/0106045 by Lee, filed Nov. 18, 2003, and entitled “Peristaltic Mixing and Oxygenation System,” each of which is incorporated herein by reference in its entirety for all purposes.

In certain embodiments, the use of a buffer, acidic material injection, alkaline material injection, and/or CO₂ transport into the gaseous headspace can be used as part of a scheme to control the CO₂ concentration and/or pH in the liquid medium. For example, dissolved CO₂ and/or pH levels can be controlled by first measuring the pH and/or dissolved CO₂ levels in the liquid medium. In some embodiments, the pH and/or dissolved CO₂ levels can be adjusted, for example, by increasing or decreasing the partial pressure of CO₂ in the gas headspace (either in direct contact with the liquid medium or separated from the liquid medium by a moveable wall), by injecting an alkaline material (e.g., a bicarbonate containing solution or other alkaline material, optionally in the form of a liquid) into the liquid medium, by injecting an acidic material (e.g., an acidic liquid) into the liquid medium, and/or by adding a buffer (e.g., a bicarbonate-based buffer) to the liquid medium. In certain embodiments, the pH and CO₂ level within the liquid medium can be adjusted independently using the strategies outlined herein. In certain embodiments, the pH and CO₂ level within the liquid medium can be adjusted independently of the osmolarity of the liquid medium. For example, in some embodiments, the pH of the liquid medium can be adjusted without adjusting the osmolarity of the liquid medium. In some embodiments, the dissolved CO₂ concentration in the liquid medium can be adjusted without adjusting the osmolarity of the liquid medium.

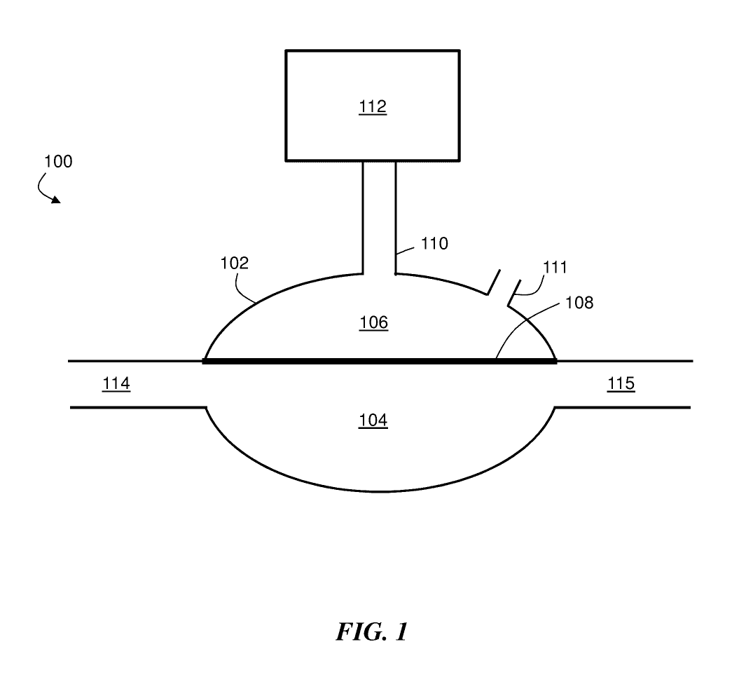

FIG. 1 is a schematic cross-sectional illustration of bioreactor system 100, according to one set of embodiments. In FIG. 1, bioreactor system comprises reactor chamber 102. Reactor chamber 102 can comprise a liquid growth medium 104. In certain embodiments, liquid growth medium 104 can contain at least one biological cell, for example, when bioreactor system 100 is used as a cell growth system. Liquid growth medium 104 can contain any type of biological cell or cell type (e.g., a prokaryotic cell and/or a eukaryotic cell). For example, the cell may be a bacterium (e.g., E. coli) or other single-cell organism, a plant cell, or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a primate cell, a bovine cell, a horse cell, a porcine cell, a goat cell, a dog cell, a cat cell, or a cell from a rodent such as a rat or a mouse. In some embodiments, the cell can be a human cell. In some embodiments, the cell may be a hamster cell, such as a Chinese hamster ovary (CHO) cell. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. In some cases, the cell may be a genetically engineered cell.

Reactor chamber 102 can comprise a gaseous headspace 106. Gaseous headspace 106 can be positioned above liquid growth medium 104 in reactor chamber 102. In certain embodiments, gaseous headspace 106 and liquid growth medium 104 can be in direct contact. In such systems, interface 108 in FIG. 1 can correspond to a gas-liquid interface. In other embodiments, gaseous headspace 106 and liquid growth medium 104 are separated by a moveable wall. For example, interface 108 can correspond to a flexible membrane. In embodiments in which such flexible membranes are employed, the membrane can be permeable to at least one gas. For example, the flexible membrane can be, in certain embodiments, permeable to oxygen and/or carbon dioxide.

In certain embodiments, the gaseous headspace can contain carbon dioxide. The concentration of carbon dioxide in the headspace can be sufficiently high, in certain embodiments, that carbon dioxide can be transported from gaseous headspace 106 to liquid growth medium 104. The rate of delivery of carbon dioxide from gaseous headspace 106 to liquid growth medium 104 and/or the equilibrium concentration of carbon dioxide and/or pH in the liquid growth medium can be adjusted, for example, by adjusting the partial pressure of carbon dioxide within gaseous headspace 106. This can be achieved, for example, by transporting gas into gaseous headspace 106 containing more or less carbon dioxide than is present within the gaseous headspace. Accordingly, in certain embodiments, reactor chamber 102 comprises a first inlet 110 connecting a source 112 of carbon dioxide gas to gaseous headspace 106. Source 112 can be any suitable source, such as a gas tank. In certain embodiments, source 112 can contain substantially pure carbon dioxide (e.g., at least about 80% carbon dioxide, at least about 90% carbon dioxide, at least about 95% carbon dioxide, or at least about 99% carbon dioxide), while in other embodiments, source 112 can contain carbon dioxide mixed with one or more other gases that can be used in association with bioreactor system 100, such as oxygen (which can be used to aerate liquid growth medium 104), nitrogen, and/or an inert gas (such as helium or argon, which might be used to actuate moveable wall 208 to produce mixing within liquid growth medium 104, as described in more detail elsewhere. Optionally, reactor chamber 102 can comprise outlet 111, which can be used to transport gas out of gaseous headspace 106. In some embodiments, changing the partial pressure of carbon dioxide can be used to control pH.

In certain embodiments, the pH of liquid growth medium 104 can be adjusted by introducing an acidic and/or alkaline material into the liquid medium. Accordingly, in some embodiments, reactor chamber 102 comprises a second inlet 114. Second inlet 114 can be connected to a source of an alkaline liquid (e.g., including alkaline liquids having a pH of greater than or equal to 7.5, greater than or equal to 8.5, greater than or equal to 9.5, greater than or equal to 11, or greater). In certain embodiments, an alkaline liquid can be transported to liquid growth medium 104 via inlet 114, which can increase the pH of liquid growth medium 104. Any suitable source of alkaline liquid can be used. In certain embodiments, the alkaline liquid can be a bicarbonate-based alkaline liquid (i.e., it can include a bicarbonate ion, HCO₃⁻). Such alkaline solutions can be formed, for example, by dissolving a bicarbonate salt (e.g., sodium bicarbonate, potassium bicarbonate, and the like) in a solvent such as water. In general, any suitable base (e.g., hydroxide bases) may be used in the alkaline liquid.

In some embodiments, the reactor chamber may operate within a set temperature range. In general the operating temperature of the reactor may be any suitable temperature that allows the growth and proliferation of prokaryotic and/or eukaryotic cells. In certain embodiments, the operating temperature of the reaction chamber is between about 20° C. and about 45° C., between about 25° C. and about 45° C., between about 30° C. and about 45° C., between about 30° C. and about 40° C., between about 33° C. and about 38° C., between about 25° C. and about 40° C., or between 20° C. and about 40° C. For example, in certain embodiments in which a reactor chamber includes eukaryotic cells (e.g., mammalian cells), the reactor chamber may have an operating temperature between about 30° C. and about 45° C. (e.g., between about 30° C. and about 40° C., between about 33° C. and about 38° C., about 37° C.). In another example, in some embodiments in which a reactor chamber includes prokaryotic cells (e.g., bacteria), the reactor chamber may have an operating temperature between about 20° C. and about 40° C. (e.g., between about 25° C. and about 40° C., between about 30° C. and about 40° C., about 30° C.)

In some embodiments, reactor chamber 102 comprises an inlet connected to a source of an acidic liquid (e.g., including acidic liquids having a pH of less than or equal to 6.5, less than or equal to 5.5, less than or equal to 4.5, less than or equal to 3, or smaller). In certain embodiments, an acidic liquid can be transported to liquid growth medium 104 via an inlet (e.g., inlet 114 or another inlet), which can decrease the pH of liquid growth medium 104. Any type of acid (e.g., an inorganic acid, an organic acid) may be used. In certain embodiments, the acid is a strong acid. The acid might also be a weak acid. For example, the acid may include hydrochloric acid (HCl), sulfuric acid (H₂SO₄), nitric acid (HNO₃), or any other suitable acid. In certain embodiments, a single inlet (e.g., inlet 114) can be used to transport base and acid (e.g., at different times) into liquid growth medium 104. In such cases, a conduit connected to inlet 114 can be bifurcated such that one upstream portion is connected to the acid source while another upstream portion is connected to the source of alkaline liquid.

Optionally, reactor chamber 102 can comprise outlet 115, which can be used to transport liquid medium out of chamber 102.

In certain embodiments, reactor chamber 102 comprises one or more sensors. For example, reactor chamber 102 can comprise a pH sensor and/or a carbon dioxide sensor. One or more sensors can be positioned or otherwise configured to be in contact with liquid growth medium 104 to measure a property of the liquid medium. One or more other sensors can be positioned or otherwise configured to be in contact with gaseous headspace 106 to measure a property of the gas within the gaseous headspace.

In certain embodiments, liquid growth medium 104 can contain a buffer, which can aid in controlling the pH of the liquid medium. A variety of types of buffers can be used. In certain embodiments, the buffer comprises a bicarbonate (i.e., HCO₃⁻) buffer. The chemical reactions associated with the bicarbonate buffer are outlined as follows:

CO₂(aq)+H₂O(l) [symbol] H₂CO₃(aq)log K_{H}=−1.41

H₂CO₃(aq) [symbol] HCO₃⁻(aq)+H⁺(aq)log Kₐ₁=−6.38

HCO₃⁻(aq) [symbol] CO₃²⁻(aq)+H⁺(aq)log Kₐ₂=−10.38

Other buffers that may be employed include, for example, sulfate-based buffers, acetate-based buffers, phosphate-based buffers, and the like.

In certain embodiments, the volume of the reactor chamber can be relatively small. For example, the reactor chamber can have a volume of equal to or less than about 50 milliliters, equal to or less than about 10 milliliters, or equal to or less than about 2 milliliters (and/or, in certain embodiments, equal to or greater than 10 microliters, equal to or greater than 100 microliters, or equal to or greater than 1 milliliter).

The reactor chamber can, in some embodiments, be configured to contain (and/or, can contain during operation of the reactor) a volume of liquid medium equal to or less than about 50 milliliters, equal to or less than about 10 milliliters, or equal to or less than about 2 milliliters (and/or, in certain embodiments, equal to or greater than 10 microliters, equal to or greater than 100 microliters, or equal to or greater than 1 milliliter).

In certain embodiments, the reactors described herein can be configured such that, during operation, the k_{L}a of carbon dioxide between the bulk of the headspace and the bulk of the liquid medium is similar to k_{L}a values successfully employed in much larger reactors. In certain embodiments, the reactor can be operated such that the k_{L}a of carbon dioxide between the bulk of the headspace and the bulk of the liquid medium is at least about 0.1 hours⁻¹ or at least about 1 hour⁻¹. In certain embodiments, the reactor can be operated such that the k_{L}a of carbon dioxide between the bulk of the headspace and the bulk of the liquid medium is less than or equal to about 15 hours⁻¹, less than or equal to about 10 hours⁻¹, or less than or equal to about 5 hours⁻¹. Those of ordinary skill in the art are familiar with the parameter k_{L}a (often referred to as the volumetric mass transport coefficient) as used to describe the transport of a gas within a reactor system, as described, for example, in V. Linek, P. Benes, and V. Vacek, “Measurement of aeration capacity of fermenters,” Chem. Eng. Technol., 1989, Vol. 12, Issue 1, pages 213-217. The “k_{L}” portion of k_{L}a generally refers to the mass transport coefficient, which encompasses all resistances to transport from the liquid to the gas. The “a” portion of k_{L}a refers to the interfacial area between the liquid and the gas. k_{L}a is the resulting product of multiplying k_{L} and a. One of ordinary skill in the art would be capable of calculating the k_{L}a value with respect to carbon dioxide for a given reactor system during operation by recreating the operating conditions, subsequently injecting pure nitrogen into the gas headspace of the reactor (the dynamic gassing method), and constructing a plot of ln(1−DCO₂) as a function of time, wherein DCO₂ is defined as:

$${DCO}_{2} = \frac{{C}_{CO 2}}{{C}_{CO 2}^{*}}$$

wherein C_{CO2} is the concentration of CO₂ at a given point in time and C*_{CO2} is the concentration of CO₂ at its saturation point in the liquid medium. After constructing such a plot, the absolute value of the slope of plot would correspond to k_{L}a with respect to CO₂. That is to say, the k_{L}a is the time constant of the decay or rise in dissolved CO₂ concentration in the medium when the partial pressure of CO₂ in the gas headspace is switched.

A variety of parameters can affect the value of k_{L}a with respect to carbon dioxide, including the mixing rate, the volume of the reactor chamber, and the partial pressure of CO₂ in the headspace. It has been unexpectedly discovered that desirable k_{L}a values with respect to CO₂ (including the k_{L}a values outlined above) can be achieved for reactors with volumes of 50 milliliters or less by using mixing rates such that substantially complete mixing (i.e., about 95% complete mixing or more) is achieved relatively slowly (e.g., in about 5 seconds or more). In addition, it can be advantageous to employ partial pressure of CO₂ in the reactor headspace of between about 0% to about 20%, between about 1% to about 20%, between about 2% to about 15%, between about 2% and about 10%, between about 3% and about 7%, or about 5%, for example, when the total headspace gas pressure is from about 0 psi to about 15 psi, 1 psi to about 15 psi, about 0 psi to about 10 psi, about 1 psi to about 10 psi, about 1 psi to about 5 psi, about 2 psi to about 4 psi, about 2.5 psi to about 3.5 psi, or at about 3 psi, relative to atmospheric pressure. In certain embodiments, it can be advantageous to set the height of the liquid medium within the reactor chamber (i.e., the distance between the top of the liquid and the bottom of the reactor chamber) to between about 0.05 inches and about 0.5 inches. It should be recognized that, in certain embodiments, other liquid heights can be employed, such as between about 0.05 inches to 2 inches, between about 0.5 inches to 2 inches, between about 0.05 inches to 1 inch, or between about 1 inch to 2 inches.

As noted above, in certain embodiments, gaseous headspace 106 and liquid growth medium 104 are in direct contact. In other embodiments, gaseous headspace 106 and liquid growth medium 104 are separated by a moveable wall. Reactors employing such arrangements are described, for example, in U.S. patent application Ser. No. 13/249,959 by Ram et al, filed Sep. 30, 2011, and entitled “Device and Method for Continuous Cell Culture and Other Reactions” and U.S. Patent Application Publication No. 2005/0106045 by Lee, filed Nov. 18, 2003, and entitled “Peristaltic Mixing and Oxygenation System,” each of which is incorporated herein by reference in its entirety for all purposes.

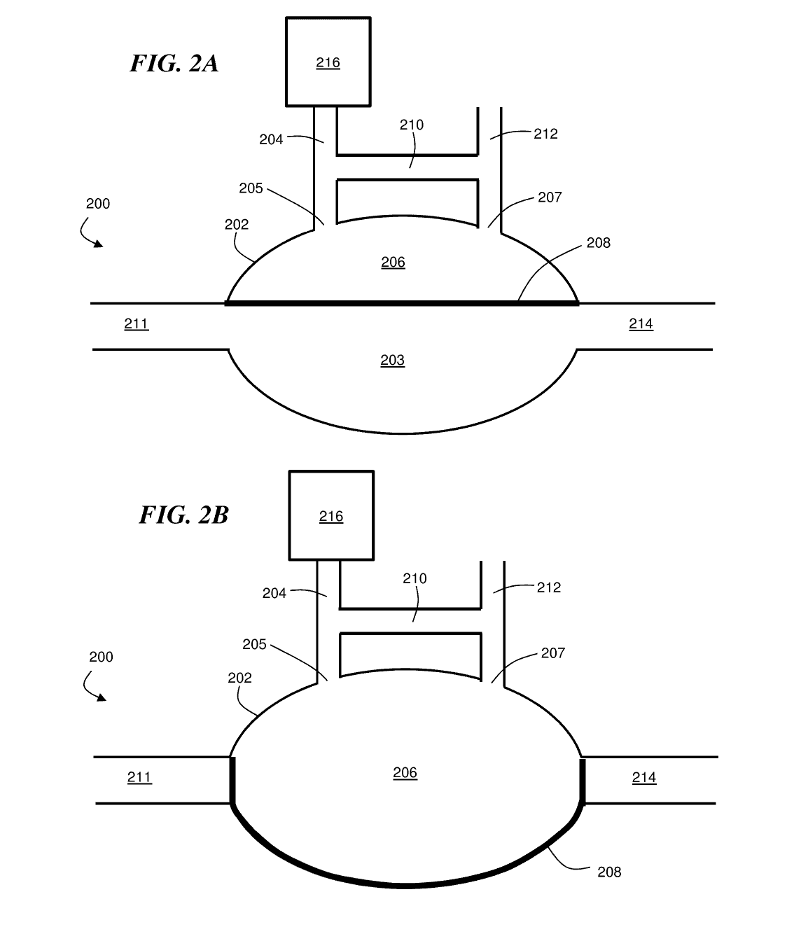

FIGS. 2A-2C are cross-sectional schematic illustrations outlining how fluid can be transported by deflecting a moveable wall into and out of a liquid sub-chamber of a reactor chamber. In FIGS. 2A-2C, reactor system 200 comprises reactor chamber 202. In certain embodiments, reactor chamber 202 in FIGS. 2A-2C corresponds to reactor chamber 102 in FIG. 1. Reactor chamber 202 can comprise a liquid sub-chamber 203. Liquid sub-chamber 203 can be configured to contain a liquid growth medium including at least one biological cell. Reactor chamber 202 can comprise, in certain embodiments, gas sub-chamber 206. Gas sub-chamber 206 can be configured to contain a gaseous headspace above the liquid growth medium within liquid sub-chamber 203.

Reactor chamber 202 can also comprise a moveable wall 208, which can separate liquid sub-chamber 203 from gas sub-chamber 206. Moveable wall 208 can comprise, for example, a flexible membrane. In certain embodiments, the moveable wall is formed of a medium that is permeable to at least one gas (i.e., a gas-permeable medium). In certain embodiments, for example, moveable wall can be permeable to oxygen gas and/or carbon dioxide gas. In such embodiments in which moveable wall 208 is permeable to a gas (e.g., oxygen and/or carbon dioxide), the gas within gas sub-chamber 206 can be transported to liquid sub-chamber 203, or vice versa. Such transport can be useful, for example, to transport oxygen gas into a liquid medium within liquid sub-chamber 203 and/or control pH by transporting carbon dioxide into or out of liquid sub-chamber 203.

Reactor system 200 can comprise, in certain embodiments, a gas inlet conduit 204, which can be configured to transport gas into gas sub-chamber 206. Gas inlet conduit 204 in FIGS. 2A-2C can correspond to the gas inlet conduit 110 illustrated in FIG. 1, in certain embodiments. The gas that is transported into gas sub-chamber 206 can originate from, for example, gas source 216. Any suitable source of gas can be used as gas source 216, such as gas cylinders. In certain embodiments, gas source 216 is a source of oxygen and/or carbon dioxide.

In some embodiments, reactor system 200 comprises gas outlet conduit 212 configured to transport gas out of gas sub-chamber 206. Gas outlet conduit 212 in FIGS. 2A-2C can correspond to the gas outlet conduit 111 illustrated in FIG. 1, in certain embodiments. In some embodiments, reactor system 200 comprises gas bypass conduit 210 connecting gas inlet conduit 204 to gas outlet conduit 212. Gas bypass conduit 210 can be configured such that it is external to reactor chamber 202, in certain embodiments. Reactor system 200 can also comprise, in certain embodiments, a liquid inlet conduit 211 and a liquid outlet conduit 214.

In certain embodiments, moveable wall 208 can be actuated such that the volumes of liquid sub-chamber 203 and gas sub-chamber 206 are modified. For example, certain embodiments involve transporting a gas from gas source 216 through gas inlet conduit 204 to gas sub-chamber 206 to deform moveable wall 208. Deformation of moveable wall 208 can be achieved, for example, by configuring reactor 200 such that gas sub-chamber 206 is pressurized when gas is transported into gas sub-chamber 206. Such pressurization can be achieved, for example, by restricting the flow of gas out of gas outlet conduit 112 (e.g., using valves or other appropriate flow restriction mechanisms) while gas is being supplied to gas sub-chamber 206.

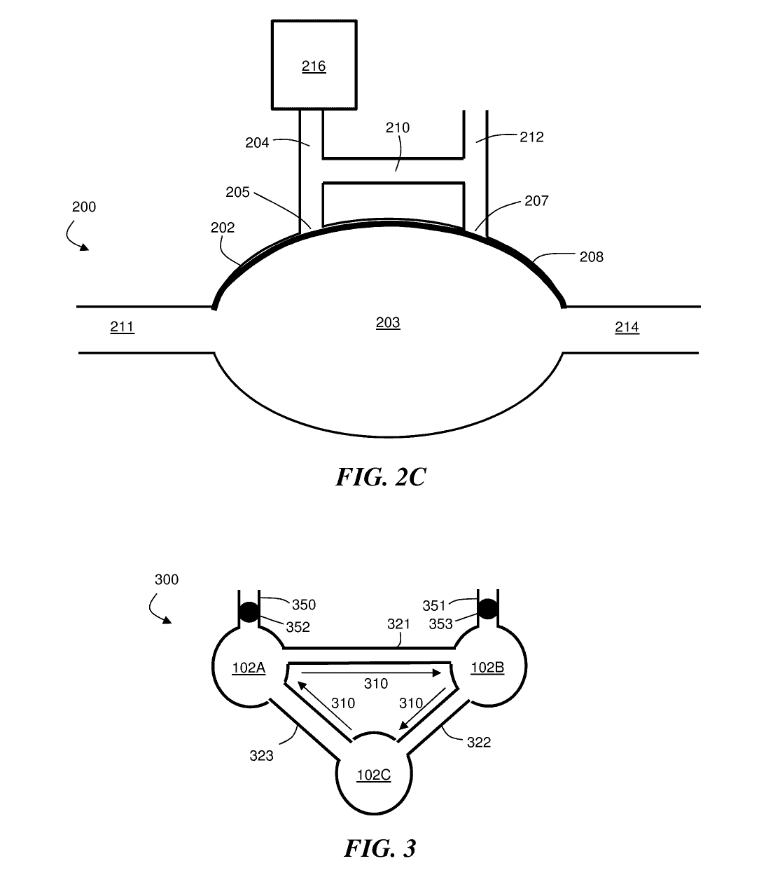

In certain embodiments, deforming moveable wall 208 can result in liquid being at least partially evacuated from liquid sub-chamber 203. For example, in FIG. 2B, moveable wall 208 has been deformed such that substantially all of the liquid within liquid sub-chamber 203 has been evacuated from reactor chamber 202. Such operation can be used to transport the liquid within liquid sub-chamber 203 to other liquid sub-chambers in other reactors, as illustrated, for example, in FIG. 3, described in more detail below.

In certain embodiments, after at least a portion of the liquid within liquid sub-chamber 203 has been removed from liquid sub-chamber 203, the supply of the gas to gas sub-chamber 206 can be reduced such that moveable wall 208 returns toward its original position (e.g., the position illustrated in FIG. 2A). In certain embodiments, moveable wall 208 will be deflected such that at least a portion of the gas within gas sub-chamber 206 is removed from the gas sub-chamber. Such gas might be removed, for example, if liquid enters liquid sub-chamber 203 from liquid inlet conduit 211, for example, from another upstream reactor, as described in more detail below.

Certain embodiments include the step of supplying gas from gas source 216 to gas sub-chamber 206 at least a second time to deform moveable wall 208 such that liquid is at least partially removed from liquid sub-chamber 203. When such gas introduction steps are performed repeatedly, moveable wall 208 can act as part of a pumping mechanism, transporting liquid into and out of liquid sub-chamber 203. Such operation is described in detail in U.S. patent application Ser. No. 13/249,959 by Ram et al, filed Sep. 30, 2011, and entitled “Device and Method for Continuous Cell Culture and Other Reactions.”

In certain embodiments in which gas is transported into gas sub-chamber 206 multiple times, gas can be transporting from the gas source through gas bypass conduit 210. Transporting gas through gas bypass conduit 210 can be performed to remove liquid from gas inlet conduit 204 without transporting the liquid to gas sub-chamber 206. For example, in certain embodiments, a first valve between gas bypass conduit 210 and gas inlet 205 can be closed and a second valve between gas bypass conduit 210 and gas outlet 207 can be closed (and any valves within gas bypass conduit 210 can be opened) such that, when gas is transported through gas inlet conduit 204, the gas is re-routed through gas bypass conduit 210, and subsequently out gas outlet conduit 212. Such operation can serve to flush any unwanted condensed liquid out of the gas inlet conduit, which can improve the performance of the gas supply methods described elsewhere herein.

In some embodiments, multiple sets of reactor chambers can be arranged (e.g., in series) such that fluidic mixing is achieved along one or more fluidic pathways. FIG. 3 is a bottom view, cross-sectional schematic diagram illustrating the liquid flow paths that can be used to establish mixing between multiple reactor chambers 102A-C connected in series, as described in U.S. patent application Ser. No. 13/249,959 by Ram et al, filed Sep. 30, 2011, and entitled “Device and Method for Continuous Cell Culture and Other Reactions.”

In FIG. 3, reactor system 300 includes a first fluidic pathway indicated by arrows 310. The first fluidic pathway can include a first reactor chamber 102A, a second reactor chamber 102B, and a third reactor chamber 102C. Reactor system 300 also includes conduits 321, 322, and 323, which can correspond to liquid inlet and/or liquid outlet conduits for reactor chambers 102A-C. For example, in FIG. 3, conduit 321 is a liquid inlet conduit for reactor chamber 102B and a liquid outlet conduit for reactor chamber 102A; conduit 322 is a liquid inlet conduit for reactor chamber 102C and a liquid outlet conduit for reactor chamber 102B; and conduit 323 is a liquid inlet conduit for reactor chamber 102A and a liquid outlet conduit for reactor chamber 102C. Of course, the flow of liquid can also be reversed such that conduits 321, 322, and 323 assume opposite roles with respect to each of reactor chambers 102A-C.

Reactor system 300 can also include a liquid input conduit 350 and a liquid output conduit 351, which can be used to transport liquid into and out of the liquid sub-chambers within reactor chambers 102A, 102B, and 102C. Valve 352 may be located in liquid input conduit 350, and valve 353 may be located in liquid output conduit 351 to inhibit or prevent to the flow of liquid out of the mixing system during operation.

In certain embodiments, the moveable walls of reactor chambers 102A-C can be actuated to transport liquid along fluidic pathway 310 (and/or along a fluidic pathway in a direction opposite pathway 310). This can be achieved, for example, by sequentially actuating the moveable walls within reactor chambers 102A-C such that liquid is transported in a controlled direction. In some embodiments, each of reactor chambers 102A-C can be configured such that they are each able to assume a closed position wherein moveable wall 208 is strained such that the volume of the liquid sub-chamber is reduced, for example, as illustrated in FIG. 2B. Peristaltic mixing can be achieved, for example, by actuating reactor chambers 102A-C such that their operating states alternate between open (FIG. 2A or FIG. 2C) and closed (FIG. 2B) configurations. In some embodiments, three patterns may be employed to achieve peristaltic pumping: a first pattern in which the liquid sub-chamber of reactor chamber 102A is closed and the liquid sub-chambers within reactor chambers 102B and 102C are open; a second pattern in which the liquid sub-chamber of reactor chamber 102B is closed and the liquid sub-chambers within reactor chambers 102A and 102C are open; and a third pattern in which the liquid sub-chamber of reactor chamber 102C is closed and the liquid sub-chambers within reactor chambers 102A and 102B are open. By transitioning among these three patterns (e.g., changing from the first pattern to the second pattern, from the second pattern to the third pattern, and from the third pattern to the first pattern, etc.) liquid can be transported among reactor chambers 102A-C in a clockwise direction (as illustrated in FIGS. 2A-2B). Of course, by re-arranging the order in which the patterns occur (e.g., by changing from the first pattern to the third pattern, from the third pattern to the second pattern, and from the second pattern to the first pattern, etc.), liquid can be transported in the counter-clockwise direction as well.

The following example is intended to illustrate certain embodiments of the present invention, but does not exemplify the full scope of the invention.

### Example

This example describes the design and operation of a reactor system integrating inventive carbon dioxide concentration and pH control methods.

The use of biologics, like monoclonal antibodies, recombinant proteins and nucleic acid based proteins, in pharmaceuticals have been well received in the last decade. Therapeutic monoclonal antibodies have revolutionized various oncology treatments because they generally have less side effects than traditional cytotoxic drugs. In 2007, there were 22 therapeutic monoclonal antibodies in the market with a value of over $17 Billion, and the market is expected to increase to around $49 Billion globally by 2013. Some of the well-known licensed monoclonal antibody treatments are Rituxan for cancer, Remicade for arthritis, Synagis for lung disease, and Herceptin for breast cancer. Currently, more than 50% of the pharmaceutical industry's pipeline portfolio consists of recombinant proteins and monoclonal antibodies and over 600 new biologics are being developed every year. Therapeutic recombinant proteins and monoclonal antibodies are produced by recombinant mammalian cells, genetically modified to overproduce the therapeutic protein. Mammalian cell lines can be preferred in many cases because they contain organelles and enzymes that can synthesize, fold and chemically modify the protein to form tertiary structure, like glycosylation, which is important for the therapeutic function of the protein. The latter process is known as post-translational modification. In some cases, where post-translational modifications of the proteins are not required, some recombinant proteins, like Insulin, can be produced in the more robust and faster growing cells like Escherichia Coli. However, most therapeutic proteins in production currently require post-translational glycosylation which can only be found in eukaryotic cells, of which about 70% are produced using the Chinese Hamster Ovary (CHO) cell line.

In spite of the rapidly growing biologics market, biopharmaceutical companies are constantly faced with pressures to reduce costs from health care providers. Moreover, the long time from discovery to market of biopharmaceuticals frequently results in early patent expiration and profit losses. Competition from generics also drives biopharmaceutical companies toward finding avenues to lower development and manufacturing costs. There also exists an urgency for pharmaceutical companies to develop a large portfolio of new drugs in order to stay ahead of rival biopharmaceutical companies. Typically, a new drug will take around 6-9 years to go through development, manufacturing, clinical trials and FDA approval before becoming available on the market. There are many benefits of shortening the time to market including longer validity of the patent when the drug is released to the market, the ability to test and develop more biologics simultaneously to increase the chances of finding a blockbuster drug and lowering the overall cost of the drug. The upstream development process for producing recombinant proteins is currently very complex and time-consuming. For the drug protein to be FDA approved, there must be consistency in the quality of the recombinant drugs produced, in particular, the glycosylation and effectiveness of therapeutic proteins must retain the same quality even for different culture batches. Since the quality of the glycosylation of the recombinant protein can be influenced by process conditions, the control and monitoring of process parameters in a bioreactor becomes very important. Moreover, since therapeutic monoclonal antibodies are used at high doses over a long period of time, in order to meet market demands, it is beneficial if the cell culture is able to produce a high product titer while maintaining the same product quality. All these requirements should be maintained during scale up and scale down of the production batches.

The upstream development of bioprocesses for the production of recombinant proteins generally include the following four stages: 1. Clone Selection, 2. Clone Stability Tests, 3. Process Development and 4. Scale Up Experiments. First, 1000 clones are grown in stationary 96 well plates to find the fastest grower and highest producer clones based on Enzyme-linked Immunosorbent Assay (ELISA) results. The selected clones (typically around 50-100 clones) are then grown in shake flasks, which is an agitated environment similar to bioreactors but without any pH, temperature, dissolved oxygen (DO) or feed rate control. Stability tests to ensure that the clones will not mutate over many generations can also be performed during this stage. From the shake flask experiments, only 4-6 clones are selected and transferred to bench scale experiments before scaling up to large scale industrial bioreactors. There is a cost-limiting factor determining the number of clones selected at this stage because bench top bioreactors and scale up experiments are very costly to run. This selection process is risky because there is evidence that clone selection from measuring growth and productivity alone as a single end point in 96 well places is not a predictor for selecting a stable cell. Moreover, shake flasks with no instrumentation or control over pH, DO or feed may not be able to select the most productive clone with a stable glycosylation profile since the product titer and quality can be affected by the actual process conditions.

Therefore, an important technology missing in conventional upstream development protocol is a miniaturized high throughput and instrumented secondary clone selection system with online sensors that is an almost exact scale down model of an industrial bioreactor with sufficient volume for offline characterization of product titer, glycosylation profiles and other important process conditions.

In the near future, biopharmaceutical companies will be looking into building cellular function models that will help elucidate the effects of feed rate, physical and chemical stresses on the cells' metabolic state. Having predictive models of the impacts of manufacturing conditions on industrially relevant cell lines would greatly accelerate the upstream process optimization by adopting a Quality by Design (QbD) approach. Often times, the overexpression of the recombinant proteins is rate limited by an enzyme whose kinetics are not well understood. Understanding the rate limiting steps affecting the productivity of cells will greatly reduce the experiments needed to find the optimal processing conditions for the recombinant cell line. The large data banks required to form a complete cellular function model require a high throughput platform that can run at a much lower operating cost than bench scale bioreactors but with the same set of instrumentations. This miniaturized biotechnology platform would have to be automated and run at least 20 experiments in parallel in order to complete the experiments in a reasonable time frame.

The Chinese Hamster Ovary (CHO) cell line is an important cell line for producing recombinant protein therapeutics, accounting for almost 70% of the biotherapy market, far exceeding other commonly used mammalian cell lines such as 3T3, BTK, HeLa and HepG2. In 2006, the worldwide sales of biopharmaceutical products produced using the CHO cell line alone exceeded $30 billion. With the burgeoning interests in expanding the range of biologics produced from CHO cells, there is an increasing demand for upstream development in high-throughput micro-bioreactors, such as microfluidic devices and well plates, specifically for recombinant CHO cell research and biotechnological process optimization. In recent years, micro-bioreactors in the form of microfluidic devices and well plates have emerged for upstream development of microbial cell lines. The development of micro-bioreactors for mammalian cell lines like CHO cells have not gained as much momentum mainly because of the added complexity when trying to adapt these microbial micro-bioreactors for the more sensitive mammalian cell lines. The design criteria for micro-bioreactors designed for mammalian cell lines are listed with yeasts and E. Coli, a bacterial cell line, in Table 1.

TABLE 1
Criteria for micro-bioreactors based upon parameters
achieved for current industrial processes.

| Parameters | Mammalian Cells | | Yeast | | E. Coli | |
|---|---|---|---|---|---|---|
| Growth rate | 0.041-0.075 | h⁻¹ | 0.5-4 | h⁻¹ | 0.1-4 | h⁻¹ |
| Doubling time | 15-24 | h | 0.5-5 | h | 0.1-4 | h |
| Cell Density | 10⁶-10⁷ | cells/mL | 200-500 | g/L wcw* | 200-500 | g/L wcw* |
| OUR | <5 | mmol/Lh | <300 | mmol/Lh | <300 | mmol/Lh |
| k_{L}a | 1-15 | h⁻¹ | 200-400 | h⁻¹ | 200-400 | h⁻¹ |
| DO | >20% | | >20% | | >20% | |
| Agitation | 50-150 | rpm | 100-3000 | rpm | 100-3000 | rpm |
| Dissolved CO₂ | 35-80 | mmHg | <5% | | <5% | |
| Temperature | 32-38° | C. | 18-30° | C. | 18-37° | C. |
| pH | 6.8-7.15 | | 4-8 | | 6-7.5 | |
| pH Control | Caustic or CO₂ addition | | Acid or caustic addition | | | |
| Cycle time | 20 | days | <7 | days | <4 | days |

Unlike bacteria or yeast cells, the growth and productivity of Chinese Hamster Ovary (CHO) cells are very sensitive to process conditions. CHO cells, like most mammalian cells, can easily undergo necrotic or apoptotic cell death under physical and chemical stresses. To give a sense of their sensitivity to shear stress, a CHO cell's shear stress tolerance is 3 orders of magnitude lower than that of an Escherichia coli (E. Coli) cell, a common type of bacteria used in biotechnology. Shear stress above 0.005 Nm⁻² have been shown to affect protein glycosylation in CHO cells due to morphological deformation of the endoplasmic reticulum, the organelle responsible for folding and glycosylation of the protein. Therefore, the micro-bioreactor should be designed to have a mixer that generates low shear stress and yet provide fast enough mixing to prevent large gradients which may cause nutrient starvation or toxicity. Moreover, the long doubling time of CHO cells (22-24 hours) generally requires a much longer culture time for CHO cells, typically 2-3 weeks long, as compared to E. Coli cultures which may last only up to 4 days due to their much shorter doubling time (about 1 hour). For long term cultures, evaporation becomes a major problem because of the high surface to volume ratio of small working volumes of micro-bioreactors. Water loss can also cause the osmolarity of the culture medium to increase to toxic levels within 5 days. Evaporation compensation strategies generally need to be employed for micro-bioreactors running long term cultures like CHO cell cultures. The longer doubling time of CHO cells also makes the culture more easily contaminated since the cells can be easily overtaken by faster growing yeast and bacteria cells. The micro-bioreactor should therefore be able to maintain sterility throughout the 10-14 days of culture duration and all process including sample removal and incubation must be performed without compromising the sterility of the growth chamber.

Since Chinese Hamster Ovary (CHO) cells are widely used for making therapeutic proteins, they have been very extensively studied and their optimal growth conditions well documented. An important process parameter for CHO cell cultures is the partial pressure of carbon dioxide, pCO₂, in the medium. The pCO₂ also affects the pH and osmolarity of the culture medium as shown in Equation 1

CO₂(aq)+H₂O(l) [symbol] HCO₃⁻(aq)+H⁺(aq) [1]

NH₃(aq)+H⁺(aa) [symbol] NH₄⁺(aq) [2]

Removal of CO₂ can increase pH and reduce osmolarity of the culture medium. A high pCO₂ in the medium can also cause the internal pH of the cells, pHᵢ, to drop since CO₂ is non-polar and hence, diffuses freely through the cell membrane. The decrease in pHᵢ can alter the cell metabolism and affect the performance of the cytostolic enzymes. Moreover, changes in the cytoplasmic pH can also alter the pH in the endoplasmic reticulum which affects post-translational protein processing, like glycosylation and secretion. Since CO₂ is a byproduct of cell metabolism, efficient stripping of CO₂ should be included in an effective CHO cell bioreactor. CO₂ gas can also be used to control pH and it is a preferred strategy over liquid acid addition because it doesn't increase the osmolarity of the medium as much as liquid additions. However, when the CHO cells reach a high density, stripping of CO₂ gas can become harder and liquid base addition will be more effective in neutralizing the acidity caused by the accumulation of CO₂ gas in the medium. For these reasons, pCO₂ control is very important for CHO cell micro-bioreactors since it affects osmolarity, pH, and glycosylation of the cells. An optimal range of pCO₂ is between 31-75 mmHg (0.04-0.10 atm) and if it exceeds 99 mmHg (0.13 atm), it can be detrimental to the growth, productivity and product quality of CHO cells.

On a separate note, mild hypoxia has been shown to cause a decrease in oxygen consumption of the cells without affecting cell growth rate, maximum cell density, recombinant protein production rate, or recombinant protein activity. The CHO cell line also shows enhanced growth in culture media with pH between 7.0 and 7.6. If the pH exceeds 8.2 or drops below 6.9, the protein glycosylation will generally be affected since the diffusion of unprotonated NH₃ at high pH (see Equation 2) and CO₂ at low pH (see Equation 1) through the cell membrane can alter the internal pH of the golgi apparatus. The glucose uptake rate, q_{GLC}, is 1.0-1.5 mMol/10¹⁰ cells/h, the oxygen consumption rate, q_{GLC}, is 1.25-1.5 mMol/10¹⁰ cells/h, and the ratio of lactose production to glucose consumption rate, Y_{LAC,GLC}, is 1.1-1.2 for CHO cells as reported in the literature. Typically for CHO cell culture, the desired osmolarity is in the range between 260-320 mOsm/kg, mimicking serum at 290 mOsm/kg. The specific death rate of mammalian cells has been shown to steadily increase as the osmolarity is increased from 320 to 375 and 435 mOsm/kg.

Bench top bioreactors are the standards for scale down models of industrial bioreactors at a scale of 1000-10,000 times smaller than industrial bioreactors. Since volume and surface area scale differently with length, the physical and chemical environment experienced by the cells even in bench top bioreactors that are geometrically identical to industrial bioreactors will be different. The physical and chemical environment of the cells can strongly affect the cells' physiology and productivity and hence should be maintained constant or within the limits of critical values during scaling. First, the gas transfer rate of O₂ and CO₂ should be sufficiently high so that the dissolved oxygen level remains above the oxygen uptake rate of the cells and waste gas like carbon dioxide are efficiently removed. Secondly, the maximum shear rate experienced by the cells should remain the same or below the critical value that affects productivity during the scaling. This can be especially important for mammalian cells like CHO due to their shear sensitivity. The circulation time is also an important parameter since it affects the frequency at which the cells experience high shear. The repeated deformation of the endoplasmic reticulum has been reported to affect protein glycosylation. Bioreactors with different chamber volumes will have very different circulation time before the cells circulate back to the tip of the impeller and hence, some bench top bioreactors are equipped with a circulation line that allows the physical environment of the cells to mimic the circulation time seen in large industrial scale bioreactors. On the other hand, the mixing rate of the micro-bioreactor must be sufficiently fast and uniform so that there is no region in the culture where the cell is nutrient starved or have a large concentration gradient. When designing scale down models of bioreactors, the energy dissipation rate should be maintained substantially constant so that the transfer of internal energy to the cell remains substantially constant.

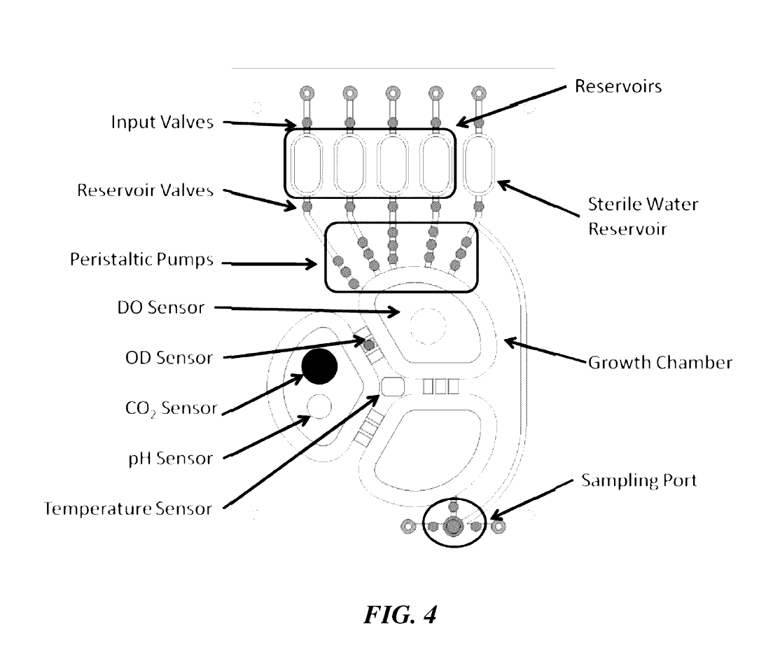

A new reactor design, referred to in this example as the Resistive Evaporation Compensated Actuator (RECA) micro-bioreactor, which is illustrated in FIG. 4, has been developed for culturing cells, including CHO cells. The reactor includes 5 reservoirs for injections, including one containing sterile water for evaporation compensation. The other four reservoirs can be used for Sodium Bicarbonate (NaHCO₃) base injections, feed, and other necessary supplements. Injection can be performed by a peristaltic pump actuated through the PDMS membrane sequentially pushing a plug of fluid into the growth chamber. In this example, the growth chamber has a volume of 2 milliliters. Uniform mixing can be obtained by pushing fluids through small channels connecting the three growth chambers, each having a volume of 1 milliliter. There is also a 10 microliter reservoir for sampling located after the growth chamber. The sampling can be performed via peristaltic pumping of 10 microliter plugs. Besides the connection to the growth chamber, the sample reservoir is also connected via a channel to the sterile water line and a clean air line. Air can be injected through the sample reservoir to eject any remaining sample into the sampling container (e.g. an Eppendorf tube), and water can be injected after that to clean the sample reservoir and remove any cell culture or cells remaining. Clean air can then be sent through the reservoir to dry the chambers so that there would no water left to dilute the next sample. This process can be repeated after each sampling step.

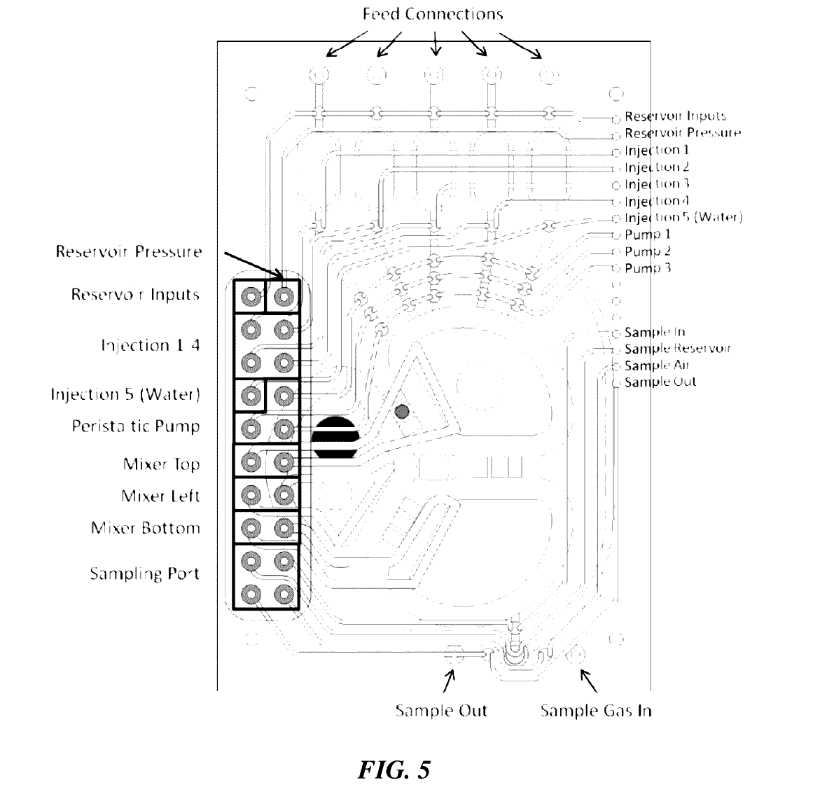

The connections from the RECA micro-bioreactor to the gas manifold are shown in FIG. 5. All reservoir input valves can share the same gas line since it is unnecessary to individually control each input valve. The reservoir pressure can be set to be 1.5 psi (1.03×10⁵ Pa), which is lower than that of the mixing pressure of 3 psi (2.06×10⁵ Pa). The reservoir pressure can be used to ensure that the input to the peristaltic pumps sees the same pressure and is unaffected by external hydrostatic pressure to ensure consistent pumping volume. The output of the reservoir, i.e. the injection valves, can be individually controlled by separate gas lines because these are the valves that determine which feed lines are being injected into the growth chamber. Next are the gas lines that control the peristaltic pumps. The mixers can have a separate input and output line in order to allow flushing of water condensation on the mixer lines, since the air coming into the mixer can be humidified to reduce evaporation of the growth culture. The growth chambers of the micro-bioreactor have large surface to volume ratios and hence, the evaporation rates are generally larger than that for larger bioreactors. Moreover, all three mixer gas lines can be designed to have the same resistance, to ensure an even mixing rate in the 3 growth chambers. The mixer gas lines can be made wider than the rest of the lines because the air is humidified, and any condensation might clog the lines if the resistance is too high. The last air lines control the valves to the sampling port. The sampling port consists of a 10 microliter sample reservoir and valves to control sampling and automated cleaning of the sampling port. The holes in the top left corner can be sealed with a polycarbonate cover and taped with double sided tape. The air lines can be connected through a group of 20 barbs located on the left bottom corner of the chip to the gas manifold.

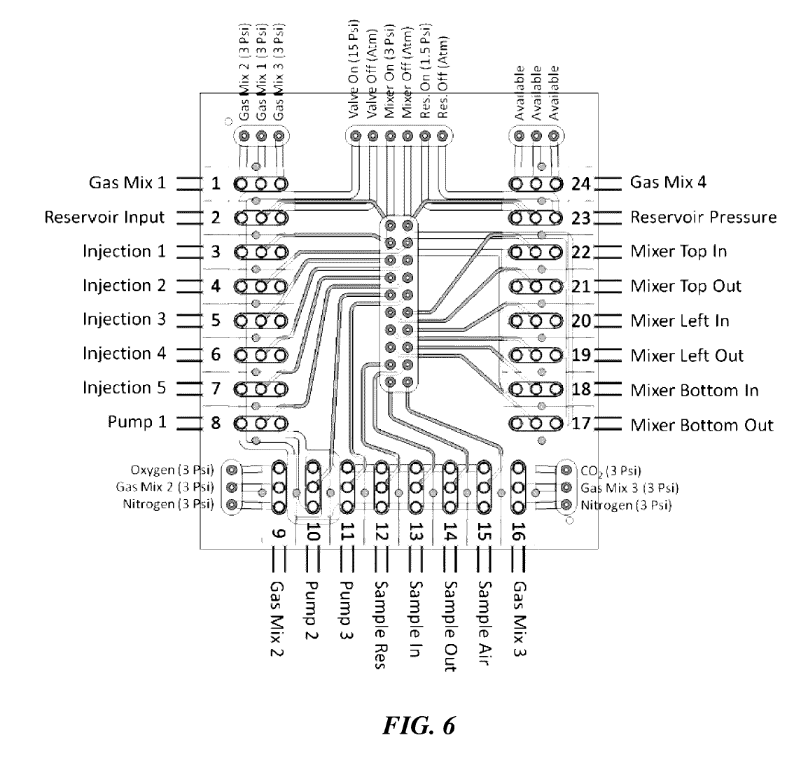

A gas manifold can be used to connect the solenoid valves to the air lines of the micro-bioreactor. The design of the gas manifold is shown in FIG. 6. The manifold in this example has 3 layers. The barb connectors to the micro-bioreactor are situated in the center of the top layer of the manifold. The middle layer routes the output of the solenoid valves to the barb connectors that connects the manifold to the micro-bioreactor. The bottom layer routes the main air lines to the inputs of the solenoid valves. Tables 2A-C lists all the valves with their numbers as shown in FIG. 6 and the gas connections for easier referencing.

### Table 2A for Valves 1-8

| Valve | Name | NO | NC |
|---|---|---|---|
| 1 | Gas Mix 1 | Gas Mix 2 (3 Psi) | Gas Mix 2 (3 Psi) |
| 2 | Reservoir Input | Valve On (15 Psi) | Valve Off (Atm) |
| 3 | Injection 1 | Valve On (15 Psi) | Valve Off (Atm) |
| 4 | Injection 2 | Valve On (15 Psi) | Valve Off (Atm) |
| 5 | Injection 3 | Valve On (15 Psi) | Valve Off (Atm) |
| 6 | Injection 4 | Valve On (15 Psi) | Valve Off (Atm) |
| 7 | Injection 5 | Valve On (15 Psi) | Valve Off (Atm) |
| | (water) | | |
| 8 | Pump 1 | Valve On (15 Psi) | Valve Off (Atm) |

### Table 2B for Valves 9-16

| Valve | Name | NO | NC |
|---|---|---|---|
| 9 | Gas Mix 2 | Nitrogen (3 Psi) | Oxygen (3 Psi) |
| 10 | Pump 2 | Valve Off (Atm) | Valve On (15 Psi) |
| 11 | Pump 3 | Valve On (15 Psi) | Valve Off (Atm) |
| 12 | Sample Reservoir | Valve On (15 Psi) | Valve Off (Atm) |
| 13 | Sample In | Valve On (15 Psi) | Valve Off (Atm) |
| 14 | Sample Out | Valve On (15 Psi) | Valve Off (Atm) |
| 15 | Sample Air In | Valve On (15 Psi) | Valve Off (Atm) |
| 16 | Gas Mix 3 | Nitrogen (3 Psi) | CO₂ (3 Psi) |

### Table 2C for Valves 17-24

| Valve | Name | NO | NC |
|---|---|---|---|
| 17 | Mixer Bottom Out | Mixer Off (Atm) | Blocked |
| 18 | Mixer Bottom In | Blocked | Mixer On (3 Psi) |
| 19 | Mixer Left Out | Mixer Off (Atm) | Blocked |
| 20 | Mixer Left In | Blocked | Mixer On (3 Psi) |
| 21 | Mixer Top Out | Mixer Off (Atm) | Blocked |
| 22 | Mixer Top In | Blocked | Mixer On (3 Psi) |
| 23 | Reservoir Pressure | Res. Off (Atm) | Res. On (1.5 Psi) |
| 24 | Gas Mix 4 | Available | Available |

In Tables 2A-2C, NO stands for Normally Open and NC stands for Normally Closed. The selection of which gas lines is normally open or normally closed can be selected to be the most common state of the valve, so that more often than not, the valve is inactive, to save energy consumption. In particular, Valve 10 (Pump 2) can be set to ‘off’ normally while all the rest of the valves are set to ‘on’ normally. There are also 4 gas mixer solenoid valves besides the solenoid valves needed for mixing and valving on the micro-bioreactor. Control of carbon dioxide (CO₂) gas concentration vs nitrogen (N₂) gas can be achieved by changing the duty cycle of Gas Mix 3 solenoid valve. Oxygen (O₂) gas concentration can be controlled via Gas Mix 2 via the same strategy. Then the two outputs can be mixed together in a 50-50 duty cycle using Gas Mix 1. Gas Mix 4 is available for use if any extra valving is needed.

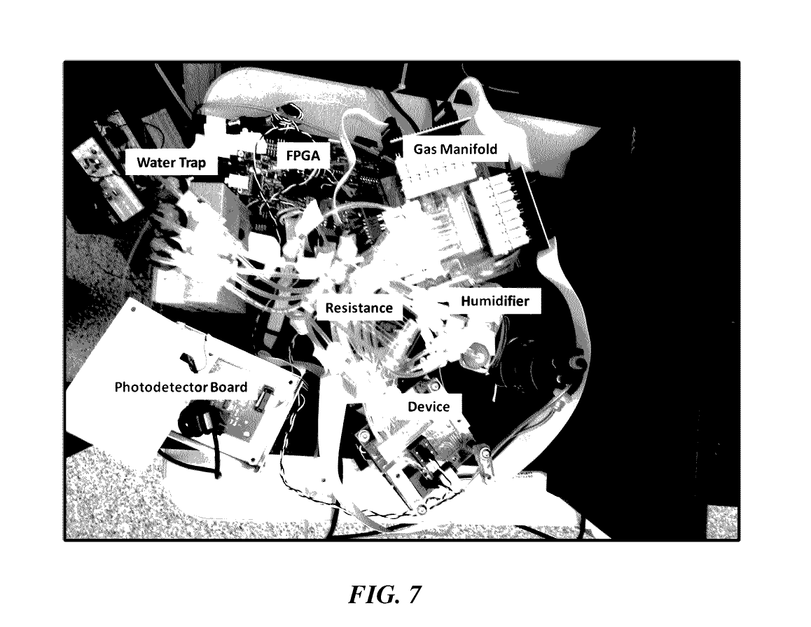

The complete setup is shown in FIG. 7. A laptop can be used to control a Field-programmable Gate Array (FPGA) board, which can control the solenoid boards, the heater board, and photo-detector board. Air lines can be connected to a pressure regulator before being connected to the gas manifold. From the gas manifold, the valve lines can be connected directly to the micro-bioreactor. The mixer in lines are connected first through an air resistance line, followed by a 45° C. local humidifier before reaching the micro-bioreactor. The mixer out lines from the micro-bioreactor are connected to the water trap, then to the air resistance lines and then only to the gas manifold.

Carbon dioxide sensors (configured to determine pCO₂) were integrated with the RECA reactor. The sensors were sensor spots from PreSens Gmbh. These sensors included gas-permeable membranes in which a short lifetime pH sensitive luminescence dye (hydroxypyrenetrisulfonic acid (HPTS)), is immobilized together with a buffer and an inert reference luminescense dye with a long lifetime. Humidified CO₂ gas permeating into the membrane changes the internal pH of the buffer and the luminescence of the HPTS. The two luminophores have overlapping excitation and emission spectra so that they can be excited with the same light source and detected with the same photodetector. The excitation source was modulated at a frequency, f_{mod}, that was compatible with the long lifetime fluorophore. Fluorophores with different lifetimes, τ, will lag behind the modulated source with a phase lag of φ, given by Equation 3

tan φ=2πf_{mod}τ [3]

The reference fluorophore will have a constant phase lag given by φ_{ref}. Since the HPTS has a very short lifetime, the phase lag will be approximately zero, φ_{ind}˜0. The real and imaginary part of the resultant emitted fluorescence from the reference and indicator dyes, with amplitude, Aₘ, and phase, φₘ, are listed in the following equations:

Aₘ cos φₘ=A_{ref} cos φ_{ref}+A_{ind} [4]

Aₘ sin φₘ=A_{ref} sin φ_{ref}+A_{ind} [5]

These equations simplify to give a linear relationship between the cotangent of the phase lag of the resultant fluorescence, cot(φₘ), and the ratio between the amplitudes of the indicator and reference fluorescence, A_{ind}/A_{ref}, since both cot(φ_{ref}) and sin(φ_{ref}) are constants.

$$cot {φ}_{m} = cot {φ}_{ref} + ( \frac{1}{sin φ ref} ) ( \frac{{A}_{ind}}{{A}_{ref}} ) \qquad [ 6 ]$$

An increase in CO₂ will result in a proportional increase of protons in the buffer region according to the three chemical equations below. The equilibrium constants are given at 20° C.

CO₂(aq)+H₂O(l) [symbol] H₂CO₃(aq)log K_{H}=−1.41 [7]

H₂CO₃(aq) [symbol] HCO₃⁻(aq)+H⁺(aa)log Kₐ₁=−6.38 [8]

HCO₃⁻(aq) [symbol] CO₃²⁻(aq)+H⁺(aq)log Kₐ₂=−10.38 [9]

The fluorescence of the indicator dye was due to the presence of unprotonated HPTS and hence an increase in pCO₂ resulted in a reduction of the fluorescence intensity of the indicator dye. The equation that relates the ratio between the amplitudes, A_{ind}/A_{ref}, to the pCO₂ is shown in Equation 10, where K is derived from the pKₐ of the HPTS and the pH of the buffer.

$$( \frac{{A}_{ind}}{{A}_{ref}} ) = ( \frac{1}{1 + {K}_{p} {CO}_{2}} ) \qquad [ 10 ]$$

The resultant phase lag, φₘ, can then be related to the partial pressure of carbon dioxide in the liquid, pCO₂, with φ₀, being the phase lag at zero pCO₂ and phiₘₐₓ, being the phase lag for the pCO₂ at saturation.

$$cot {φ}_{m} = cot {φ}_{max} + ( \frac{cot {φ}_{o} - cot {φ}_{max}}{1 + {K}_{p} {CO}_{2}} ) \qquad [ 11 ]$$

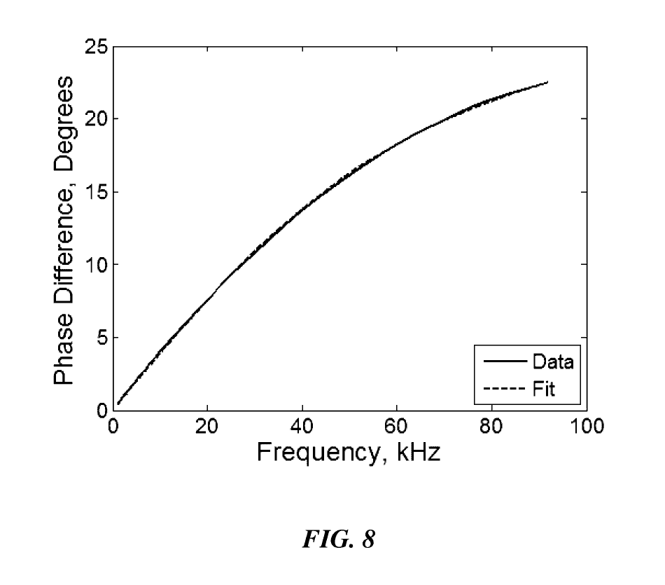

First, the optimal modulation frequency, f_{mod}, of the excitation light at 430 nm should be determined. The emission of the sensor was detected at a wavelength of 517 nm. Since the indicator had a decay time in the ns range, and the reference had a decay time in the microsecond range, the f_{mod} was swept between 500 Hz and 30 MHz to find the optimum frequency. CO₂-free Sodium Hydroxide (NaOH) solution was prepared by dissolving NaOH pellets in doubly distilled water after boiling and purging with nitrogen (N₂) gas. For the high pCO₂ concentration solution, a 1 M NaHCO₃ solution was used. Measurement of the phase lag was performed for the 1 M NaHCO₃ solution and then subtracted with CO₂ free solution for the entire frequency range. The frequency at which the difference in phases, Δφ, is the largest was chosen as the optimal modulation frequency, f_{mod}. If one assumes the response time of the reference dye to be 50 microsecond and that of the indicator to be 50 ns, then assuming that at zero point, A_{ind}˜A_{ref}, and at saturation, A_{ind}<<A_{ref}, one can theoretically model the phase difference, Δφ, as a function of the modulation frequency, f_{mod}, with Equation 12. The results are plotted in FIG. 8.

$${Δφ}_{m} = {φ}_{ref} ( {f}_{mod} ) - {cot}^{- 1} ( \frac{cos {φ}_{ref} ( {f}_{mod} ) + cos {φ}_{ind} ( {f}_{mod} )}{sin {φ}_{ref} ( {f}_{mod} ) + sin {φ}_{ind} ( {f}_{mod} )} ) \qquad [ 12 ]$$

After obtaining the optimal modulation frequency, the sensors were calibrated at that frequency with solutions with different pCO₂ concentrations at 37° C., the operating temperature. For the calibration the CO₂ free solution as described earlier and dilutions of 1 M NaHCO₃ solutions will be used. To calculate the pCO₂ in each of the standard solutions, Equations 14 to 16 can be used. The equilibrium constants listed in the equations are valid for a temperature of 20° C. In order to translate the equilibrium constants for 37° C., the Gibbs free energy of the reaction can be calculated according to the following equation.

ΔG₀=−RT ln K_{eq} [13]

From, Equation 13, it can be seen that T₁ ln K_{eg} (T1)=T₂ ln K_{eq}(T₂). Hence, the three chemical equilibrium equations can be rewritten with the new equilibrium constants at 37° C.

CO₂(aq)+H₂O(l) [symbol] H₂CO₃(aq)log K_{H}=−1.33 [14]

H₂CO₃(aq) [symbol] HCO₃⁻(aq)+H⁺(aq)log Kₐ₁=−6.03 [15]

HCO₃⁻(aq) [symbol] CO₃²⁻(aq)+H⁺(aq)log Kₐ₂=−9.81 [16]

From these equations, one can calculate pCO₂ using the method outlined in Muller, et al., “Fluorescence Optical Sensor for Low Concentrations of Dissolved Carbon Dioxide,” Analyst, 121(March):339-343, 1996. The partial pressures of carbon dioxide, pCO₂, are listed for different concentrations of NaHCO₃ solutions in Table 3.

TABLE 3
Calculated dissolved carbon dioxide concentrations
from NaHCO₃ solutions at 25° C. from measured
pH values and known concentrations of NaHCO₃.

| NaHCO₃ (M) | pH | H₂CO₃ [symbol] (M) | pCO₂ (atm) |
|---|---|---|---|
| 0.001 | 7.51 | 6.89 × 10⁻⁵ | 0.0018 |
| 0.003 | 8.02 | 6.69 × 10⁻⁵ | 0.0017 |
| 0.01 | 8.35 | 1.05 × 10⁻⁴ | 0.0027 |
| 0.03 | 8.34 | 3.22 × 10⁻⁴ | 0.0083 |
| 0.1 | 8.38 | 9.80 × 10⁻⁴ | 0.0252 |
| 0.3 | 8.31 | 3.50 × 10⁻³ | 0.0888 |

[symbol]

indicates data missing or illegible when filed

The solutions were freshly mixed and stored in a sealed vial before and during the measurement, and the vial remained sealed and stirred to decrease the response time of the sensor. The sensor can be calibrated with the CO₂-free NaOH standard solutions and the rest of the NaHCO₃ solutions in increasing concentration with an LED modulated at the optimal frequency measured in the previous experiment. The calibration graph can be fitted to Equation 11. The values of φ₀ can be obtained from the CO₂-free measurement. φₘₐₓ and K can be obtained from best fit parameters.

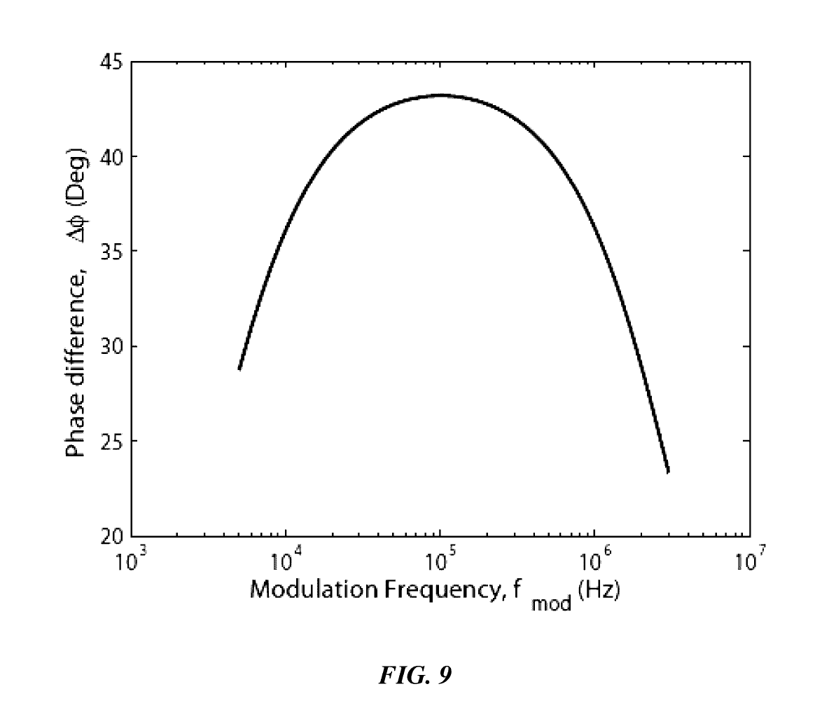

The CO₂ sensor was illuminated by an LED (430 nm) modulated at frequencies between 1 kHz and 100 kHz to obtain the optimal modulation frequency. Since there was an electronic low pass filter that cuts off the frequency at 100 kHz in the circuit, the highest modulation frequency that was possible for the system is 93 kHz. The signal obtained from the photodiode was then compared with the reference signal and the phase lag between the two signals were obtained. This measurement was performed on 1 mM NaHCO₃ solution and then repeated with 1M NaHCO₃ solution. The phase difference between the two measurements was then plotted as a function of frequency and shown in FIG. 9. The data obtained can be fitted to Equation 6 to obtain the lifetimes of the reference and indicator dyes. From the fitting, the lifetime of the reference dye was measured to be 2.5 microseconds (which is close to the literature value of ˜5 microseconds), and the lifetime of the indicator dye was measured to be 312 ns, which is similar to the literature value of 173-293 ns. From the measurements, the optimal modulation frequency that gives the highest sensitivity was the highest modulation frequency of the electronic system, which was around 93 kHz. The modulation frequencies chosen for this sweep were selected to be prime numbers to avoid noise in the measurements due to harmonics of electrical noise sources in the background.

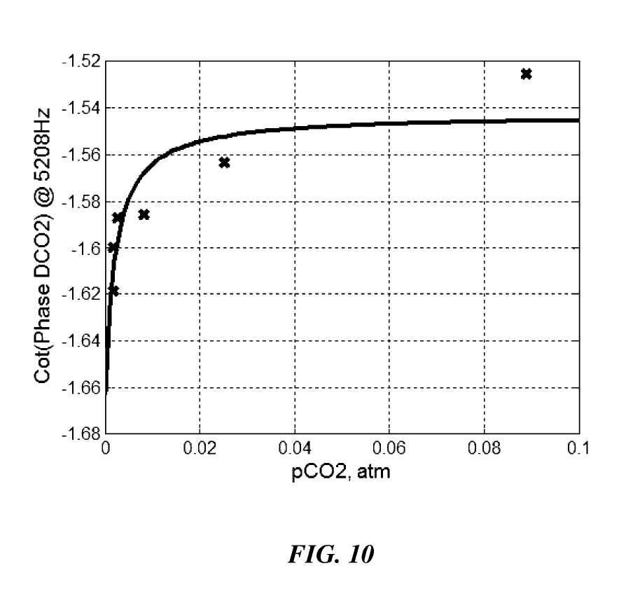

The CO₂ sensor was calibrated with Sodium Bicarbonate (NaHCO₃) solutions of different concentrations to represent solutions with different levels of dissolved CO₂ as listed in FIG. E8. The solutions were fleshly mixed and then sealed. Just before the measurement, the pH of the solution was measured to determine the concentration of dissolved CO₂. Once the solution was injected into the micro-bioreactor with the CO₂ sensor, the phase measurement was allowed to reach steady state. The results are plotted in FIG. 10. The data was fitted to Equation 11 with the value of K=3.43×10³. The measured maximum phase lag, φₘₐₓ, was 147° and phase lag at zero dissolved CO₂ concentration, φ₀, was 149°.

In order for the micro-bioreactor to have the same aeration rate as a large scale bioreactor, the gas transfer rate (k_{L}a) of the new RECA micro-bioreactor was characterized both for oxygen and carbon dioxide. This characterization was performed once the optimal mixing time was determined for each resistance line because the gas transfer rate, k_{L}a, a time constant, is related to both the diffusivity of the gas species through the PDMS membrane and the liquid as well as the mixing rate in the liquid. The higher the diffusivity and mixing rate, the faster the transport of gas species to the bottom of the chamber where the sensors are located. A sufficient gas transfer rate of oxygen is necessary to ensure that the cells have sufficient oxygen and do not enter into a hypoxic state. Using parameters that provide a proper gas transfer rate of carbon dioxide can ensure that pH control is similar to that observed in large scale bioreactors.

To determine k_{L}a for oxygen, an experiment was performed using the dynamic gas method since, for coalescent liquids like our system, the steady state and dynamic gassing values of k_{L}a were comparable, as described in V. Linek, P. Benes, and V. Vacek, “Measurement of aeration capacity of fermenters,” Chem. Eng. Technol., 1989, Vol. 12, Issue 1, pages 213-217. In the experiments described in this example, the gas in the head space of the mixer was switched from a medical gas mixture (21% O₂, 5% CO₂ and balance N₂) to pure nitrogen (100% N₂). The differential equation that describes the gas transfer relationship of oxygen is given by Equation 17, where C represents the dissolved oxygen concentration in the liquid, C* is the saturation concentration of oxygen in the liquid, and OUR refers to the oxygen uptake rate in the liquid (e.g. the oxygen uptake rate of biological cells or a molecule that absorbs oxygen in the liquid).

$$\frac{d C}{d t} = {k}_{L} a ( {C}^{*} - C ) - OUR \qquad [ 17 ]$$

Solving the differential equation above, an exponential relationship was obtained for the concentration of dissolved oxygen, C, as a function of time with OUR=0.

C(t)=C*(1−e^{-k}^{_{L}}^{at}) [18]

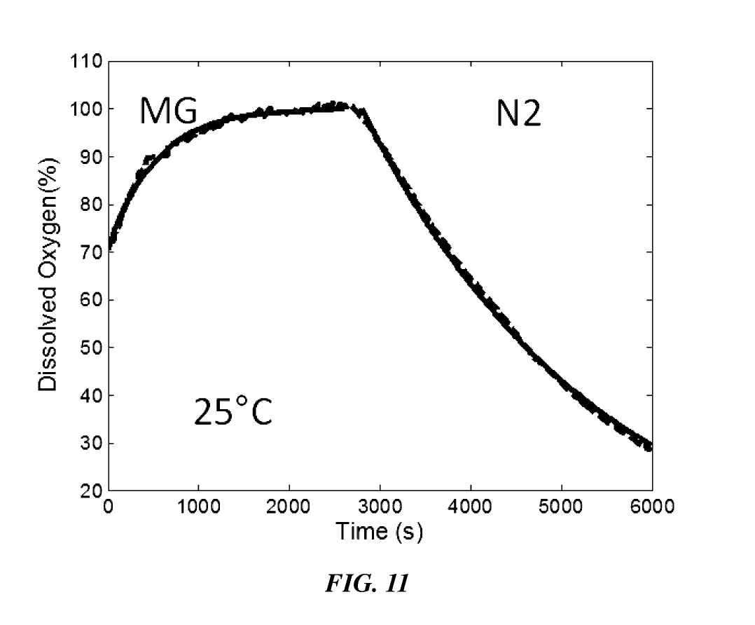

The results measured by the oxygen sensor utilizing the dynamic gassing method are shown in FIG. 11 for Resistance Line 1 at an optimal mixing cycle time of 12 seconds. From the measurement, the k_{L}a obtained when oxygen was diffusing from the head space through the membrane into the liquid (i.e. when medical gas mixture is in the head space), was 6.9±0.1 hours⁻¹. When the gas was switched to pure nitrogen, oxygen was being purged from the system by low concentration of oxygen in the head space, and the gas transfer rate of purging was measured to be 1.37±0.04 hours⁻¹. As a comparison, the gas transfer rate for oxygen for a 15,000 L bioreactor is 2-3 hours⁻¹ and 15 hours⁻¹ for a 2 L bioreactor.

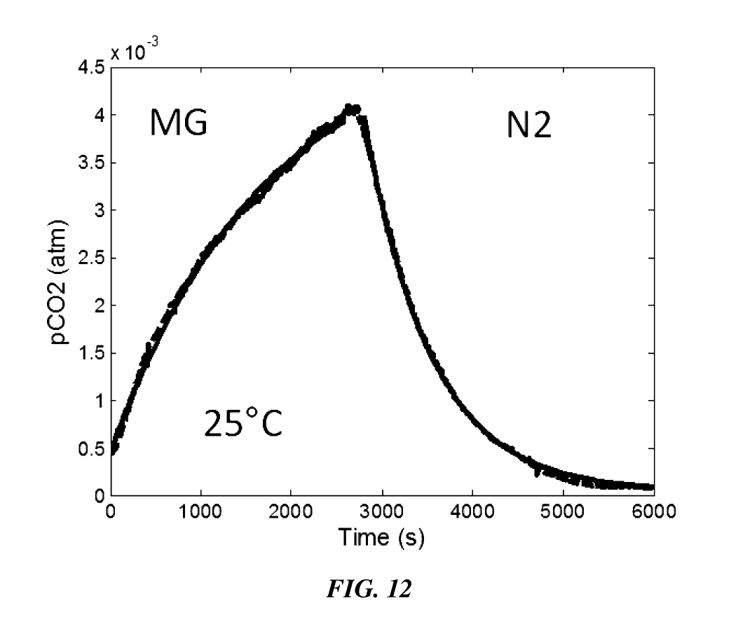

In the same experiments, the CO₂ gas transfer rate was also measured, since the medical gas mixture contained CO₂ gas as well. The results are shown in FIG. 12 for Resistance Line 1. Two exponential graphs were fitted to the data to obtain the time constant which is the inverse of k_{L}a. From the an exponential fit to the data, the gas transfer rate, k_{L}a of CO₂ from the medical gas mixture was 2.14±0.07 hours⁻¹ and the gas transfer rate from the liquid into the pure nitrogen gas head space was 4.93±0.04 hours⁻¹. As a comparison, the gas transfer rate of CO₂, k_{L}a, for a 15,000 L bioreactor is 0.2-0.4 hours⁻¹ and for a 2 L bioreactor is 5-6 hours⁻¹.

Experimental results showing control of pH using CO₂ gas variation in the headspace through a PDMS membrane (triangles) are shown in FIG. 12. The lines represent the best fit to the data. The experiment was performed with CD CHO (Invitrogen) as the liquid medium. A 70 μm thick PDMS membrane was used as a gas-permeable wall. The CO₂ in the gas headspace was mixed with O₂ and He by modulating the duty cycles of solenoid valves to generate different proportions of each gas in the headspace. The pH was measured using an optical pH sensor (PreSens) located at the bottom of the liquid chamber. The pH sensor was pre-calibrated with pH buffers and the pH measurements were compared with a standard pH probe. The liquid medium was agitated by the flexing membrane to facilitate gas transfer. As a comparison, a medical gas mixture (75% N₂, 20% O₂ and 5% CO₂) was used in the headspace as well. In FIG. 12, the data point for the medical gas mixture is shown as a circle.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A bioreactor system, comprising:
a reactor chamber having a volume of equal to or less than about 50 milliliters and containing a liquid growth medium including at least one biological cell and a buffer and a gaseous headspace containing carbon dioxide above the liquid growth medium;
a first inlet connecting a source of carbon dioxide gas to the gaseous headspace; and
a second inlet connecting a source of an alkaline liquid to the liquid growth medium.

2. A bioreactor system, comprising:
a reactor chamber having a volume of equal to or less than about 50 milliliters and containing a liquid growth medium including at least one biological cell and a buffer and a gaseous headspace containing carbon dioxide above the liquid growth medium;
a first inlet connecting a source of carbon dioxide gas to the gaseous headspace; and
a sensor within the reactor chamber configured to determine the concentration of carbon dioxide and/or pH within the liquid growth medium.

3. The bioreactor system of claim 1, comprising a sensor within the reactor chamber configured to determine the concentration of carbon dioxide within the liquid growth medium.

4. The bioreactor system of claim 1, wherein an aspect ratio of the reactor chamber is less than about 10.

5. The bioreactor system of claim 2, wherein the sensor is in direct contact with the liquid growth medium.

6. The bioreactor system of claim 1, wherein the reactor chamber comprises a moveable wall.

7. The bioreactor system of claim 6, wherein the moveable wall separates the gaseous headspace and the liquid growth medium.

8. The bioreactor system of claim 6, wherein the moveable wall is permeable to at least one gas.

9. The bioreactor system of claim 8, wherein the gas is oxygen and/or carbon dioxide.

10. The bioreactor system of claim 1, wherein the biological cell is selected from the group consisting of single-cell organisms, plant cells, and animal cells.

11. The bioreactor system of claim 10, wherein the single-cell organism is a bacterium, a protozon, a trypanosome, an amoeba, a yeast cell, or algae.

12. The bioreactor system of claim 1, wherein the biological cell is from a multicellular organism.

13. The bioreactor system of claim 1, wherein the biological cell is a mammalian cell selected from the group consisting of primate cells, bovine cells, horse cells, porcine cells, goat cells, dog cells, cat cells, rodent cells, human cells, and hamster cells.

14. The bioreactor system of claim 1, wherein the biological cell is a cardiac cell, a fibroblast, a keratinocyte, a hepatocyte, a chondrocyte, a neural cell, a osteocyte, a muscle cell, a blood cell, an endothelial cell, an immune cell, or a stem cell.

15. The bioreactor system of claim 1, wherein the biological cell is a genetically engineered cell.

16. The bioreactor system of claim 1, wherein the source of carbon dioxide gas comprises substantially pure carbon dioxide.

17. The bioreactor system of claim 1, wherein the alkaline liquid contains bicarbonate ions.

18. The bioreactor system of claim 1, wherein the alkaline liquid has a pH of greater than or equal to 7.5.

19. The bioreactor system of claim 1, comprising a third inlet connecting a source of acidic material to the liquid growth medium.

20. The bioreactor system of claim 19, wherein the acidic material has a pH of less than or equal to 6.5.

21. The bioreactor system of claim 1, wherein the gaseous headspace and the liquid growth medium are in direct contact.

22. The bioreactor system of claim 1, wherein the cell comprises a Chinese hamster ovary (CHO) cell.

23. The bioreactor system of claim 1, wherein the reactor chamber is configured to contain a volume of the liquid medium of equal to or greater than 10 microliters and less than about 50 milliliters.

24. A method of operating a bioreactor, comprising:
providing a reactor chamber having a volume of equal to or less than about 50 milliliters and containing a liquid growth medium including at least one biological cell and a gaseous headspace containing carbon dioxide above the liquid growth medium; and
operating the reactor such that the kLa of carbon dioxide between the headspace and the bulk of the liquid medium is at least about 0.1 hours' and less than about 10 hours′.

25. A method of operating a bioreactor, comprising:
providing a reactor chamber having a volume of equal to or less than about 50 milliliters, the reactor chamber containing:
a liquid growth medium including at least one biological cell, and a gaseous headspace containing carbon dioxide above the liquid growth medium;
transporting a gas containing carbon dioxide to the gaseous headspace; and
transporting an alkaline liquid to the liquid growth medium.

26. The method of claim 25, comprising operating the reactor such that the kLa of carbon dioxide between the headspace and the bulk of the liquid medium is at least about 0.1 hours' and less than about 10 hours′.

27. The method of claim 24, comprising transporting an acidic material to the liquid growth medium.

28. The method of claim 25, wherein the alkaline liquid contains bicarbonate ions.

29. The method of claim 24, wherein the k_{L}a of carbon dioxide between the headspace and the bulk of the liquid medium is less than or equal to about 10 hour⁻¹.

30. The method of claim 24, wherein the reactor chamber contains a volume of the liquid medium of equal to or greater than 10 microliters and less than about 50 milliliters.
